(19) United States
(12) Patent Application Publication
(10) Pub. No.: US 2008/0242943 A1
(43) Pub. Date: Oct. 2, 2008

(54) SYSTEM AND METHOD OF PATIENT MONITORING AND DETECTION OF MEDICAL EVENTS

(76) Inventors: Paul E. Cuddihy, Ballston Lake, NY (US); Mark D. Osborn, Castleton, NY (US)

Correspondence Address: GENERAL ELECTRIC COMPANY;GLOBAL RESEARCH, PATENT DOCKET RM. BLDG. K1-4A59, NISKAYUNA, NY, 12309, US

(21) Appl. No.: 11/692,794

(22) Filed: Mar. 28, 2007

(51) Int. Cl. A61B 5/00

(52) U.S. Cl. 600/300

(57) ABSTRACT

A system of patient health condition monitoring includes a device configured to measure a health parameter of a patient and a computer. The computer is programmed to receive an input based on the measured health parameter, determine a first moving average value for a first period of time based on the measured health parameter and determine a second moving average value for a second period of time based on the measured health parameter, the second period of time different than the first period of time. The computer is further programmed to calculate a difference between the first and second moving average values and store the difference in computer memory.

### BACKGROUND OF THE INVENTION

The present invention relates generally to patient monitoring and, more particularly, to a system and method of monitoring a health parameter of a patient.

Patient healthcare often includes monitoring a patient's well-being over time to determine or predict a future health problem or event. Carefully watching a patient health parameter often indicates whether a certain treatment is successful or not successful or whether an undesirable health condition may occur.

For example, a physician or other medical staff member may monitor the weight of a heart failure patient. A weight gain over 5 pounds, for example, may be indicative of an impending decompensation event. One general test that a physician may use to anticipate a decompensation event includes determining whether the patient has gained weight of 5 pounds over three days. Such weight gain may be indicative of water retention and, therefore, an impending decompensation event.

However, variations in day-to-day weights and subjectivity regarding the reference “normal” weight can make this calculation less than objective. In addition, a high number of false alerts occurring with inappropriate algorithms increase costs and time required by medical staff and personnel.

Therefore, it would be desirable to design a system and method that increases objectivity in and reduces false alerting in patient health parameter monitoring.

### BRIEF DESCRIPTION OF THE INVENTION

The present invention is a directed system and method for patient healthcare monitoring that overcomes the aforementioned drawbacks. A pair of moving average values of a measured health parameter for different time periods are calculated. The difference between the pair of moving average values is determined and stored.

Therefore, according to an aspect of the present invention, a system of patient health condition monitoring including a device configured to measure a health parameter of a patient and a computer. The computer is programmed to receive an input based on the measured health parameter, determine a first moving average value for a first period of time based on the measured health parameter and determine a second moving average value for a second period of time based on the measured health parameter, the second period of time different than the first period of time. The computer is further programmed to calculate a difference between the first and second moving average values and store the difference in computer memory.

According to another aspect of the present invention, a method of patient monitoring includes calculating a short-term moving average value based on a measured patient health parameter and calculating a long-term moving average value based on the measured patient health parameter. The method also includes comparing the short-term moving average value to the long-term moving average value and storing a result of the comparison to database on a computer readable storage medium.

In accordance with yet another aspect of the present invention, a computer readable storage medium having stored thereon a computer program comprising instructions that, when executed by a processor, cause the computer to acquire a value indicating a health state of a patient, calculate a fast moving average value based on the value, and calculate a slow moving average value based on the value. The instructions further cause the computer to calculate a difference between the fast moving average value and the slow moving average value and store the difference in computer readable memory.

Various other features and advantages of the present invention will be made apparent from the following detailed description and the drawings.

### BRIEF DESCRIPTION OF THE DRAWINGS

The drawings illustrate one preferred embodiment presently contemplated for carrying out the invention.

In the drawings:

FIG. 1 is a flowchart of a patient monitoring system according to an embodiment of the present invention.

FIG. 2 is a flowchart of a patient monitoring system according to another embodiment of the present invention.

FIG. 3 is a flowchart of a patient monitoring system according to another embodiment of the present invention.

FIG. 4 is a graph showing measured patient health parameters according to an embodiment of the present invention.

FIG. 5 is a graph showing measured patient health parameters removed therefrom according to an embodiment of the present invention.

FIG. 6 is a block diagram of a patient monitoring network system according to an embodiment of the present invention.

### DETAILED DESCRIPTION OF THE PREFERRED EMBODIMENT

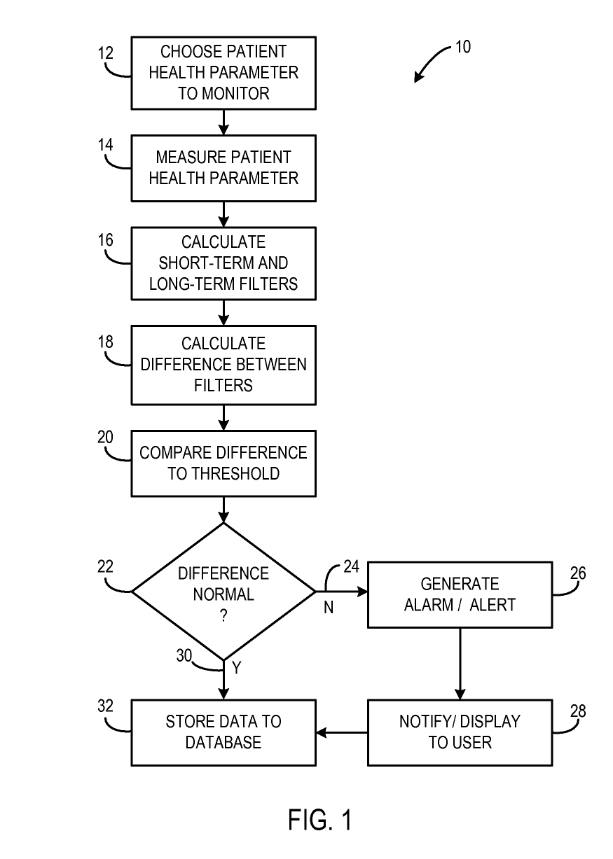

FIG. 1 shows a patient monitoring technique 10 according to an embodiment of the present convention. Technique 10 begins with choosing a health parameter of a patient to monitor 12. The patient health parameter is typically chosen by a physician for monitoring over a period of time such that indications of the future events may be detected. For example, it has been found that acute decompensation in heart failure patients, such as a failure of the heart to maintain adequate blood circulation, may be anticipated if a patient gains a significant amount of weight over a short period of time. Accordingly, the physician may require measurement of the patient's weight for monitoring over time. It is contemplated, however, that other health parameters may be measured for monitoring. For example, a physician may require the measurement and monitoring of health parameters such as systolic or diastolic blood pressure, pulse, blood sugar, and the like. An amount of change in these parameters within a time frame or an absolute change in these parameters with respect to a threshold may indicate an impending event of required critical care.

Technique 10 includes the measurement of a health parameter of the patient 14. In a preferred embodiment, the patient is allowed to measure the health parameter in the comfort of his own home. In this manner, the patient is not required to visit the hospital or to stay in the hospital while tracking the health parameter. Preferably, the patient health parameter is measured on a device that the patient already owns or can acquire. For instance, when the health parameter is weight, the patient may already have a scale in his home. Alternatively, he may acquire the scale either from the physician or on his own. If, for example, the health parameter to be measured is not measured on a device typically found in the home, the patient may require assistance from the physician to acquire such a device.

After measurement of the health parameter 14, a short-term or fast moving average and a long-term or slow moving average are calculated 16. In one embodiment, the short-term and long-term moving averages are calculated as a simple moving average of the most recent measurements over a period of time, such as 3 days for the short-term moving average and 60 days for the long-term moving average. In another example, the short-term and long-term moving averages are calculated using an exponential moving average. For a 3-day exponential moving average, for example, an average determined on a previous day is multiplied by 2 and added to the measurement of the current day, and an average of the sum equals the 3-day exponential moving average value. It is contemplated that other moving average methods may be used to determine the short-term and long-term moving averages such as a rolling moving average or a weighted moving average. One skilled in the art would recognize that embodiments of the present invention may be effective using low-pass filters, different forms of the moving average, or other convolutions or filters commonly used to analyze time series data.

After the short-term moving average and long-term moving average are calculated 16, a difference 18 between them is calculated. In a preferred embodiment, the long-term moving average is subtracted from the short-term moving average. The difference is compared to a threshold at 20. As described above, a physician may desire patient monitoring to determine if a patient has gained a significant amount of weight over a short period of time. In one example, if the difference between the short-term and long-term moving averages crosses a threshold of approximately 5 pounds above the patient's basis weight, it may indicate water retention in the patient, which may be an indicator that a decompensation event will shortly occur. In another example, a physician may desire to know if a patient has lost a significant amount of weight over a short period of time. Accordingly, the difference between the short-term and long-term moving averages may cross a threshold of approximately 5 pounds below the patient's basis weight. In an alternative embodiment, the long-term moving average is subtracted from the most recently measured patient health parameter instead of the short-term moving average.

Technique 10 determines if the difference between the short-term moving average and the long-term moving average is in a normal range or has passed a threshold limit at 22. If the difference is not within a normal region 24, an alarm or alert is generated 26. In one embodiment, the physician or other medical staff member, such as a nurse, is notified 28 when the alert is generated. For example, a nurse at a central nursing station may be notified on a workstation display that the patient has fallen out of the normal range. The nurse may, in turn, notify the physician.

After the alert is generated 26 and an appropriate medical staff member is notified 28 or if the difference between the short-term moving average and the long-term moving average is within a normal region 30, technique 10 stores various data regarding the monitor patient health parameter to a database 32. The database may store one value or all values associated with monitoring the patient. For example, the database may store only measured health parameters such that short-term moving averages, long-term moving averages, the difference between the moving averages, and alerts are determined on-the-fly. Alternatively, the database may store all parameters measured and calculated via technique 10.

In one embodiment of the present invention, steps 16-32 of technique 10 are performed via device measuring the patient health parameter. In this manner, after the device generates an alert 26, data regarding the alert and any previously stored data may be transmitted directly to a workstation display at a central facility responsible for monitoring the patient for notifying the medical staff member.

In another embodiment, a central facility acquires or retrieves the measured health parameter 14 and performs steps 16-32. In one example, the device is directly connected to a computer or similar device at the central facility, and the device is programmed to send to the measured health parameter to the computer at the central facility when the measurement is taken. In another example, it is contemplated that the patient may record his own measurement and relay that measurement to the central facility. For example, an automated telephone system may allow the patient to enter the measurement over the telephone, or a computer may allow the patient to enter the measurement over the Internet.

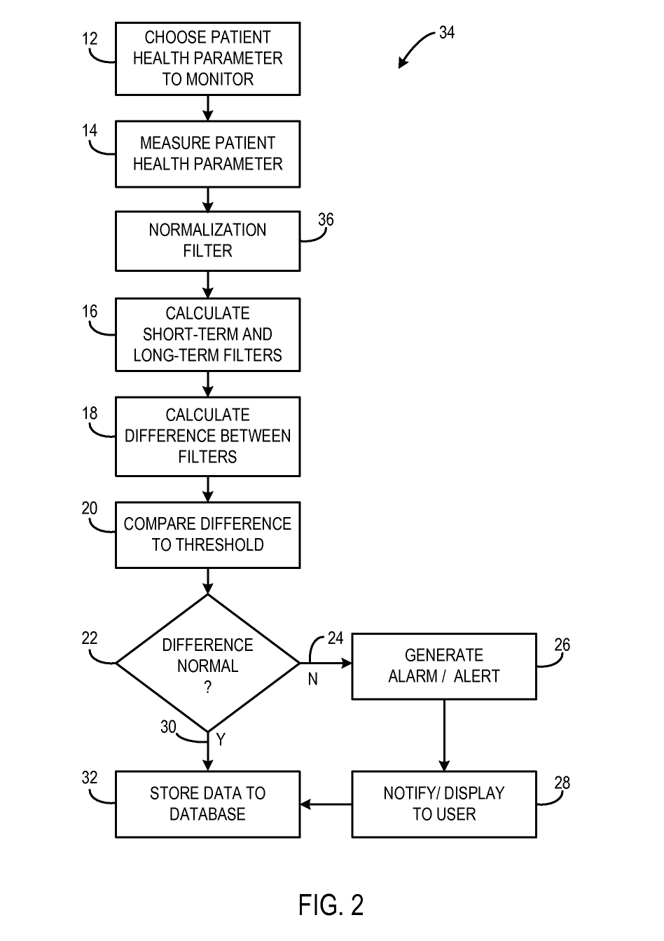

FIG. 2 shows a patient monitoring technique 34 according to another embodiment of the present invention. Technique 34 includes steps 12-32 of technique 10 as shown and described with regard to FIG. 1. Patient monitoring technique 34 additionally includes a normalization filter 36 that may be performed after measuring the health parameter at 14. The normalization filter includes normalizing the measured health parameter according to a time of day or other outlier determination. In a preferred embodiment, the patient would measure the health parameter consistently from day-to-day, such as before a morning shower or before going to bed at night. However, if the patient breaks away from this routine, for example, measuring a parameter after eating breakfast rather than before a shower, a weight or blood sugar value may be higher as a result. Accordingly, the measured health parameter may be modified or removed at 36 after retrieving the measured health parameter. For example, if, over time, a patient is typically 0.8 pounds heavier after breakfast, the filtering at 36 may automatically subtract such weight from the measured health parameter.

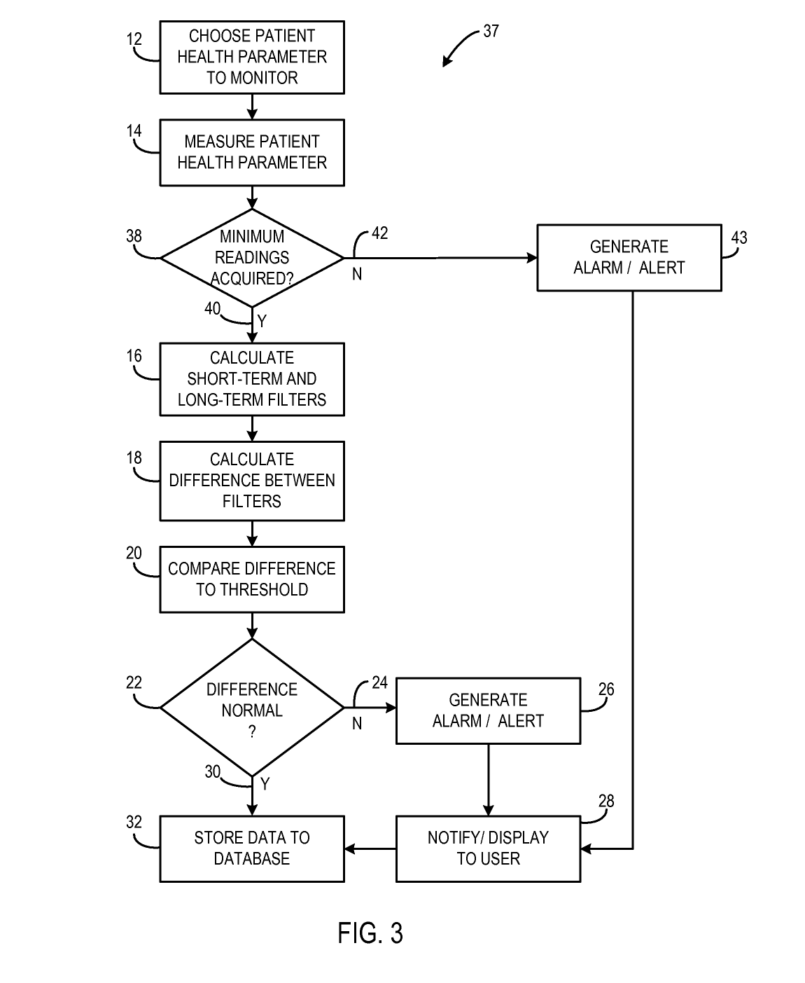

FIG. 3 shows a patient monitoring technique 37 according to another embodiment of the present invention. Technique 37 includes steps 12-32 of technique 10 as shown and described with regard to FIG. 1. Patient monitoring technique 37 additionally determines whether a minimum number of health parameter readings for a time period have been acquired 38 before calculating the short-term and long-term moving averages. For example, the short-term moving average might require three measured parameters in the past five days for calculation thereof. As another example, the long-term moving average may require ten measured parameters in the past one hundred days for calculation thereof. If a minimum number of health parameter readings have been acquired for each moving average 40, the short-term and long-term moving averages may then be calculated at 18. If a minimum number of health parameter readings have not been acquired for each moving average 42, then an alert may be generated 43 for notifying a physician or other medical staff member at 28, if desired, that there is not enough data for a reliable calculation. It is contemplated that technique 37 may also include the normalization filter 36 of technique 34 shown in FIG. 2. Accordingly, filtered normalization may be performed after measuring the health parameter at 14.

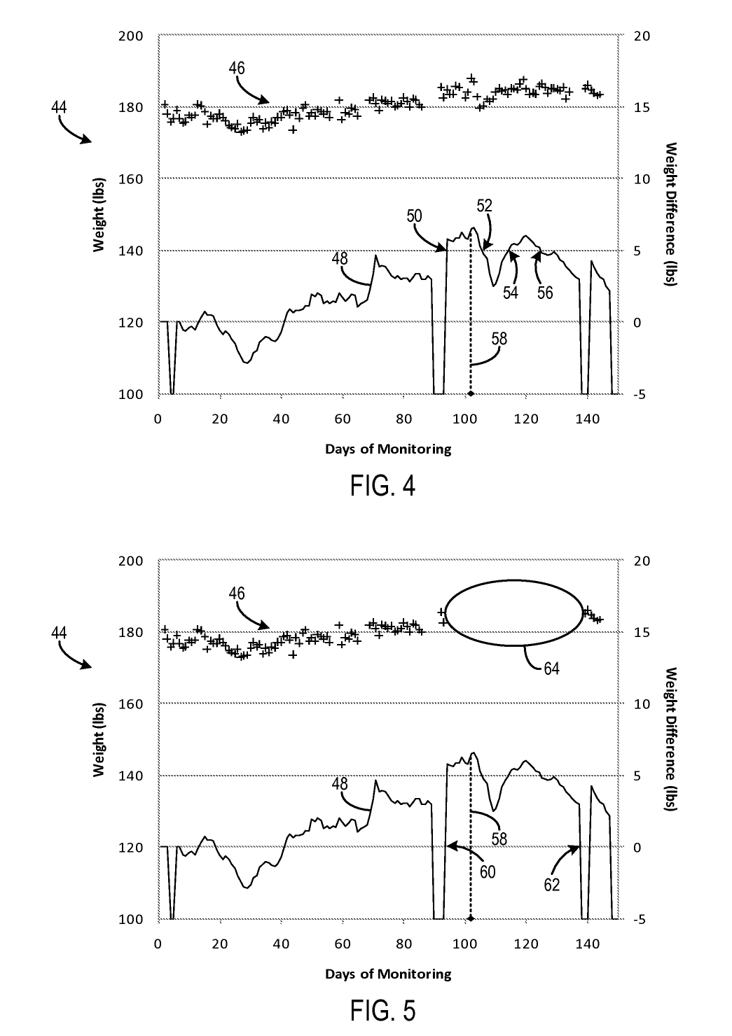

FIG. 4 shows an example of a graph 44 that may be displayed to a user. Graph 44 shows an overlay of measured daily patient health parameters 46 and a curve 48 showing the difference of the short-term and long-term moving averages over time from a sample patient. For each day a measured health parameter was received that the difference between the short-term and long-term was greater than a predetermined threshold of, for example, 5 pounds, as shown between points 50 and 52 and between points 54 and 56, techniques 10, 34 and/or 37, as described above, would generate an alarm. As shown in FIG. 4, curve 48 shows that, for a decompensation event 58 at day one hundred and two, the difference of the short-term and long-term moving averages was greater than the threshold value of 5 pounds each day for nine days prior to the decompensation event 58. Accordingly, an alert would have been generated for each of the nine days prior to the decompensation event 58.

It has been found that, once an event such as a decompensation event has occurred, the patient monitoring techniques 10, 34 and/or 37 described above may more accurately detect a future event if data surrounding the occurred event is removed from the long-term moving average. In a preferred embodiment, data immediately before and after the occurred event is removed until the moving average difference equals zero. FIG. 5 shows an area 64 where data from the database between the points 60 and 62 has been removed. In one embodiment, curve 48 showing the calculated difference between points 60 and 62 related to the removed data 64 is not modified such that all values of the moving average difference/threshold calculation appear in the patient history.

The short-term and long-term moving averages as well as the threshold for a particular measured health parameter for a certain individual or class of people may be a dynamic value. For example, it may be determined from a particular patient that a certain threshold of weight gained over a short period of time does not adequately predict an impending event. Alternatively, a “standard” period of time typically used for all cases in either the short-term or long-term moving averages might be found to be insufficient to adequately predict an impending event for a specific individual or for a particular group of people. Accordingly, optimization of the short-term and long-term moving averages and threshold over time may be required to satisfactorily predict an impending event and reduce false alerts.

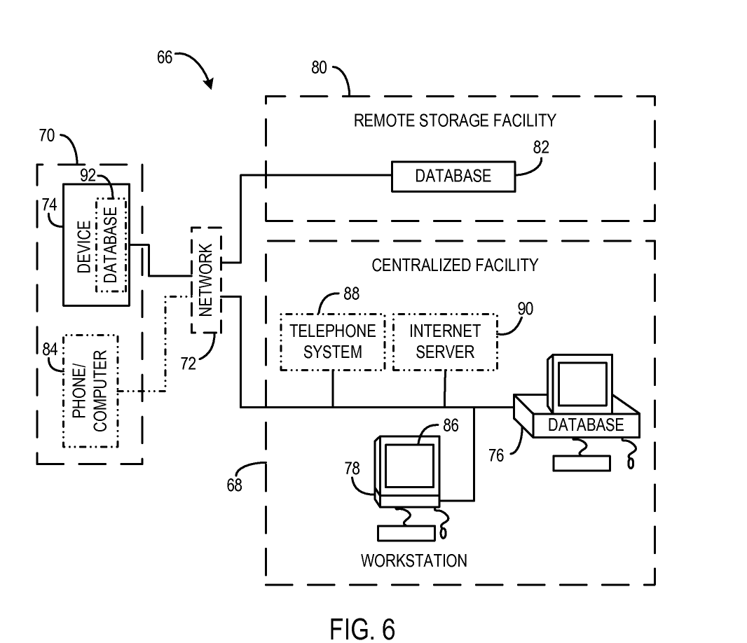

FIG. 6 shows an overview block diagram of a patient monitoring network system 66. System 66 includes a centralized facility 68 and a remote location 70. In one embodiment, centralized facility 68 includes a hospital, a clinic, or other medical facility and/or location where medical staff may monitor a patient, and remote location 70 includes a patient's home, office, or hospital room. The remote location 70 is connected to the centralized facility 68 through a communications link 72, such as a network of interconnected server nodes. This network of interconnected nodes may be a secure, internal, intranet, telephone, or a public communications network, such as the internet. Furthermore, the nodes may be interconnected through wired or wireless protocols. Although a single centralized facility 68 is shown and described, it is understood that the present invention contemplates the use of multiple centralized facilities, each capable of communication with each other.

A device 74 for measuring a patient health parameter is located at the remote location 70. Device 74 is preferably directly connected to centralized facility 68. In a one embodiment of the present invention, device 74 communicates the health parameter it measures either to a workstation 78 or to a database 76 at the centralized facility 68. If the health parameter is communicated to workstation 78, it is contemplated that workstation 78 may communicate the measure health parameter to database 76 for storage. In another embodiment, a remote storage facility 80 is connected to centralized facility 68 via communications link 72 and is configured to communicate with, receive, and store the measured health parameter from device 74 in a database 82. Accordingly, workstation 78 may connect to either database 76 or database 82 to retrieve data therefrom.

In another embodiment, device 74 is a stand-alone unit that does not connect directly to centralized facility 68 or remote storage facility 80. Accordingly, a patient may measure a health parameter on device 74 and manually add the measured health parameter to either database 76 or database 82. In one example, a telephone or computer 84 located at remote location 70 allows the patient to connect to a telephone system 88 or an internet server 90, respectively. The telephone system 88 or internet server 90 allows the patient to log in to the centralized facility 68 and input data related to the patient into the patient's records.

In one embodiment, workstation 78 is programmed with a patient monitoring technique described above in FIGS. 1-3. In this manner, workstation 78 may generate an alert for a user, such as a physician, nurse, or other medical staff member, logged into workstation 78 when a moving average difference triggers a threshold alert. The alert may be displayed to the user on a display 86 of workstation 78. In addition, workstation 78 may generate an audible alert. Workstation 78 may also generate for a user a table or graph, such as graph 44 of FIG. 4, showing recorded data for a particular patient. In this manner, a physician or other medical practitioner may review a patient's progress so far or for a particular period. It is contemplated that workstation 78 may have a patient's recorded data stored thereon or may retrieve the patient's recorded data from database 76 or database 82.

In another embodiment, device 74 is programmed with a patient monitoring technique described above in FIGS. 1-3. In this manner, device 74 may measure and calculate data related to a patient health parameter and store such data in a database 92 coupled to device 74. An alert generated for a user, such as a physician, nurse, or other medical staff member, logged into workstation 78 may be transmitted to workstation 78 when a moving average difference triggers a threshold alert. The alert may be displayed to the user on a display 86 of workstation 78.

A technical contribution for the disclosed method and apparatus is that it provides for a computer implemented system and method of monitoring a health parameter of a patient.

Therefore, according to an embodiment of the present invention, a system of patient health condition monitoring including a device configured to measure a health parameter of a patient and a computer. The computer is programmed to receive an input based on the measured health parameter, determine a first moving average value for a first period of time based on the measured health parameter and determine a second moving average value for a second period of time based on the measured health parameter, the second period of time different than the first period of time. The computer is further programmed to calculate a difference between the first and second moving average values and store the difference in computer memory.

According to another embodiment of the present invention, a method of patient monitoring includes calculating a short-term moving average value based on a measured patient health parameter and calculating a long-term moving average value based on the measured patient health parameter. The method also includes comparing the short-term moving average value to the long-term moving average value and storing a result of the comparison to database on a computer readable storage medium.

In accordance with yet another embodiment of the present invention, a computer readable storage medium having stored thereon a computer program comprising instructions that, when executed by a processor, cause the computer to acquire a value indicating a health state of a patient, calculate a fast moving average value based on the value, and calculate a slow moving average value based on the value. The instructions further cause the computer to calculate a difference between the fast moving average value and the slow moving average value and store the difference in computer readable memory.

The present invention has been described in terms of the preferred embodiment, and it is recognized that equivalents, alternatives, and modifications, aside from those expressly stated, are possible and within the scope of the appending claims.

What is claimed is:

1. A system of patient health condition monitoring comprising:
a device configured to measure a health parameter of a patient;
a computer programmed to:
receive an input based on the measured health parameter;
determine a first moving average value for a first period of time based on the measured health parameter;
determine a second moving average value for a second period of time based on the measured health parameter, the second period of time different than the first period of time;
calculate a difference between the first and second moving average values; and
store the difference in computer memory.

2. The system of claim 1 wherein the computer is further programmed to:
compare the difference to a range of non-alarm values; and
trigger an alarm if the difference falls outside of the range of non-alarm values.

3. The system of claim 2 wherein the computer is further programmed to display the alarm to a user.

4. The system of claim 1 wherein the device is located remotely from the computer.

5. The system of claim 4 wherein the device is connected to the computer via a communications link.

6. The system of claim 5 wherein the computer is programmed to receive the input directly from the device over the communications link.

7. The system of claim 6 were in the device is further configured to automatically communicate the measured health parameter to the computer over the communications link.

8. The system of claim 4 wherein the computer is programmed to receive the input via a telephone system.

9. The system of claim 1 wherein the first period of time is 3 days and wherein the second period of time is 60 days.

10. The system of claim 1 wherein the health parameter is one of a weight, a blood pressure, a pulse, and a blood sugar of the patient.

11. The system of claim 1 wherein the first and second moving average values are determined based on one of a simple moving average, a rolling average, a weighted moving average, and an exponential moving average.

12. A method of patient monitoring comprising:
calculating a short-term moving average value based on a measured patient health parameter;
calculating a long-term moving average value based on the measured patient health parameter;
comparing the short-term moving average value to the long-term moving average value; and
storing a result of the comparison to database on a computer readable storage medium.

13. The method of claim 12 further comprising comparing the result of the comparison to a threshold.

14. The method of claim 13 further comprising alerting a medical staff member if the result of the comparison crosses a threshold.

15. The method of claim 13 wherein the measured patient health parameter is weight and wherein the threshold is approximately 5 pounds.

16. The method of claim 12 further comprising removing stored results from the database immediately before and after a decompensation event while the results are greater than or equal to zero.

17. The method of claim 12 further comprising automatically receiving the measured patient health parameter from a device configured to measure the patient health parameter.

18. The method of claim 12 wherein the step of calculating the short-term moving average comprises calculating a 3-day average including the measured patient health parameter and measured patient health parameters from two preceding days; and
wherein the step of calculating the long-term moving average comprises calculating a 60-day average including the measured patient health parameter and measured patient health parameters from fifty-nine preceding days

19. The method of claim 18 further comprising adjusting the measured patient health parameter if the patient health parameter is inconsistently measured.

20. A computer readable storage medium having stored thereon a computer program comprising instructions that, when executed by a processor, cause the computer to:
acquire a value indicating a health state of a patient;
calculate a fast moving average value based on the value;
calculate a slow moving average value based on the value; calculate a difference between the fast moving average value and the slow moving average value; and
store the difference in computer readable memory.

21. The computer readable storage medium of claim 20 wherein the instructions that cause the computer to calculate the fast and slow moving average values comprise instructions that cause the computer to calculate the fast and slow moving average values based on at least one of a moving average protocol, an exponential protocol, a weighted average protocol, and a rolling average protocol.

22. The computer readable storage medium of claim 20 wherein the instructions further cause the computer to connect to a device located remotely from the computer readable storage medium; and
wherein the instructions that cause the computer acquire the value of causing the computer to automatically acquire the value directly from the device.

23. The computer readable storage medium of claim 20 wherein the instructions that cause the computer to calculate the fast moving average value comprise instructions that cause the computer to calculate the fast moving average value for three days; and
wherein the instructions that cause the computer to calculate the slow moving average value comprise instructions that cause the computer to calculate the slow moving average value for sixty days.
